(19) United States
(12) Patent Application Publication
(10) Pub. No.: US 2025/0120604 A1
(43) Pub. Date: Apr. 17, 2025

(54) SPHYGMOMANOMETER AND METHOD FOR MEASURING BLOOD PRESSURE

(71) Applicant: OMRON HEALTHCARE Co., Ltd., Kyoto (JP)

(72) Inventor: Yukiya SAWANOI, Kyoto (JP)

(21) Appl. No.: 18/990,476

(22) Filed: Dec. 20, 2024

Related U.S. Application Data
(63) Continuation of application No. PCT/JP2023/018720, filed on May 19, 2023.

(30) Foreign Application Priority Data
Sep. 16, 2022 (JP) 2022-148241

(51) Int. Cl. A61B 5/025; A61B 5/022

(52) U.S. Cl. CPC A61B 5/025; A61B 5/022; A61B 5/02225

(57) ABSTRACT

A sphygmomanometer includes a blood pressure measurement unit configured to measure a user's blood pressure based on a pulse wave signal in a pressurization process of pressurizing a cuff pressure indicating an inner pressure of a cuff worn on a part of the user to be measured. The blood pressure measurement unit sets a pressurization speed in a predetermined period of a pressurization process to be slower than a pressurization speed in a period other than the predetermined period in the pressurization process. The predetermined period is set based on a timing at which the amplitude of the pulse wave signal during the pressurization process is maximum or the timing at which the cuff pressure during the pressurization process is an average blood pressure of the user. The sphygmomanometer further includes a determination unit that determines arrhythmia of the user based on the pulse wave signal in the pressurization process.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/018720, filed May 19, 2023, which application claims priority to Japanese Patent Application No. 2022-148241, filed Sep. 16, 2022, which applications are incorporated herein by reference in their entireties.

### TECHNICAL FIELD

The present disclosure relates to a sphygmomanometer and a method for measuring a blood pressure.

### BACKGROUND ART

A technique for detecting atrial fibrillation using a signal acquired in the process of measuring a blood pressure is known from the prior art. For example, a sphygmomanometer according to Patent Document 1 (JP 2020-192322 A) determines atrial fibrillation through data of an interval time between pulse signals of pressurization stage measurement data and data of an interval time between pulse signals of depressurization stage measurement data.

### CITATION LIST

### Patent Literature

Patent Document 1: JP 2020-192322 A

### SUMMARY OF INVENTION

### Technical Problem

As a method for determining arrhythmia using a sphygmomanometer based on the oscillometric method, a method is known that determines arrhythmia such as atrial fibrillation based on a pattern of pulse wave intervals acquired during a pressurization process or a depressurization process of a cuff pressure. For example, a rising point or a maximum point of a pulse wave signal for each beat is detected as a feature point, and an interval between a current beat and a beat one beat before is calculated as a pulse wave interval. In order to accurately calculate the pulse wave interval, the amplitude of the acquired pulse wave signal is preferably great. Therefore, in order to increase the accuracy of the arrhythmia determination based on the pattern of the pulse wave interval, it is preferable to acquire a large number of pulse wave signals having a great amplitude.

In one aspect, an object of the present disclosure is to provide a sphygmomanometer and a method for measuring a blood pressure capable of accurately determining arrhythmia by acquiring many pulse wave signals having a great amplitude at the time of blood pressure measurement.

### Solution to Problem

In one example of the present disclosure, a sphygmomanometer includes a blood pressure measurement unit configured to measure a blood pressure of a user based on a pulse wave signal in a pressurization process of pressurizing a cuff pressure indicating an inner pressure of a cuff worn on a part of the user to be measured. The blood pressure measurement unit sets a pressurization speed in a predetermined period of a pressurization process to be slower than a pressurization speed in a period other than the predetermined period in the pressurization process. The predetermined period is set based on a timing at which the amplitude of the pulse wave signal during the pressurization process is maximum or the timing at which the cuff pressure during the pressurization process is an average blood pressure of the user. The sphygmomanometer further includes a determination unit configured to determine arrhythmia of the user based on the pulse wave signal in the pressurization process.

According to the above-described configuration, an arrhythmia can be accurately determined by acquiring many pulse wave signals having great amplitudes at the time of blood pressure measurement by the pressurization measurement method.

In another example of the present disclosure, the blood pressure measurement unit sets, as the predetermined period, a period from a timing at which the amplitude of the pulse wave signal during the pressurization process is maximum until after elapse of a specified time.

According to the above-described configuration, a pulse wave signal having a great amplitude can be efficiently acquired at the time of blood pressure measurement by the pressurization measurement method.

In another example of the present disclosure, the sphygmomanometer further includes a storage unit configured to store related information in which the cuff pressure and the pulse wave signal obtained at the time of past blood pressure measurement of the user are associated with each other. The blood pressure measurement unit extracts the cuff pressure at a timing at which the amplitude of the pulse wave signal in the pressurization process is maximum at the time of past blood pressure measurement of the user based on the related information, and sets, as a predetermined period, a period from a timing at which the cuff pressure in the pressurization process at the time of current blood pressure measurement of the user reaches the extracted cuff pressure until after elapse of a specified time.

According to the above-described configuration, the pulse wave signal having a great amplitude can be efficiently acquired based on the past measurement result of the user at the time of blood pressure measurement by the pressurization measurement method.

In another example of the present disclosure, the sphygmomanometer further includes a storage unit configured to store the systolic blood pressure and the diastolic blood pressure of the user obtained at the time of past blood pressure measurement of the user. The blood pressure measurement unit calculates the average blood pressure based on the systolic blood pressure and the diastolic blood pressure, and sets, as a predetermined period, a period from a timing at which the cuff pressure in the pressurization process at the time of current blood pressure measurement of the user until reaches the average blood pressure until after elapse of a specified time.

According to the above-described configuration, the pulse wave signal having a great amplitude can be efficiently acquired based on the past measurement result of the user at the time of blood pressure measurement by the pressurization measurement method.

In another example of the present disclosure, the blood pressure measurement unit sets the pressurization speed in the predetermined period to zero. According to the above-described configuration, more pulse wave signals having great amplitudes can be acquired at the time of blood pressure measurement by the pressurization measurement method.

In another example of the present disclosure, a sphygmomanometer includes a blood pressure measurement unit configured to measure, after a pressurization process of pressurizing a cuff pressure indicating an inner pressure of a cuff worn on a part of the user to be measured up to a pressure greater than a specified pressure, a blood pressure of the user based on a pulse wave signal in a depressurization process of depressurizing the cuff pressure. The blood pressure measurement unit sets a depressurization speed in a predetermined period of the depressurization process to be lower than a depressurization speed in a period other than the predetermined period of the depressurization process. The predetermined period is set based on a timing at which the amplitude of the pulse wave signal is maximum in the depressurization process or the timing at which the cuff pressure during the depressurization process is an average blood pressure of the user. The sphygmomanometer further includes a determination unit configured to determine arrhythmia of the user based on the pulse wave signal in the depressurization process.

According to the above-described configuration, an arrhythmia can be accurately determined by acquiring many pulse wave signals having great amplitudes at the time of blood pressure measurement by the depressurization measurement method.

In another example of the present disclosure, the blood pressure measurement unit sets, as the predetermined period, a period from a timing at which the amplitude of the pulse wave signal during the depressurization process is the maximum until after elapse of a specified time.

According to the above-described configuration, a pulse wave signal having a great amplitude can be efficiently acquired at the time of blood pressure measurement by the depressurization measurement method.

In another example of the present disclosure, the sphygmomanometer further includes a storage unit configured to store related information in which the cuff pressure and the pulse wave signal obtained at the time of past blood pressure measurement of the user are associated with each other. The blood pressure measurement unit extracts the cuff pressure at a timing at which the amplitude of the pulse wave signal in the depressurization process is maximum at the time of past blood pressure measurement of the user based on the related information, and sets, as a predetermined period, a period from a timing at which the cuff pressure in the depressurization process at the time of current blood pressure measurement of the user reaches the extracted cuff pressure until after elapse of a specified time.

According to the above-described configuration, the pulse wave signal having a great amplitude can be efficiently acquired based on the past measurement result of the user at the time of blood pressure measurement by the depressurization measurement method.

In another example of the present disclosure, the sphygmomanometer further includes a storage unit configured to store the systolic blood pressure and the diastolic blood pressure of the user obtained at the time of past blood pressure measurement of the user. The blood pressure measurement unit calculates the average blood pressure based on the systolic blood pressure and the diastolic blood pressure, and sets, as a predetermined period, a period from a timing at which the cuff pressure in the depressurization process at the time of current blood pressure measurement of the user reaches the average blood pressure until after elapse of a specified time.

According to the above-described configuration, the pulse wave signal having a great amplitude can be efficiently acquired based on the past measurement result of the user at the time of blood pressure measurement by the depressurization measurement method.

In another example of the present disclosure, in the pressurization process, the blood pressure measurement unit estimates the systolic blood pressure and the diastolic blood pressure of the user, estimates the average blood pressure based on the estimated systolic blood pressure and diastolic blood pressure, and sets, as a predetermined period, a period from a timing at which the cuff pressure during the depressurization process reaches the average blood pressure until after an elapse of a specified time as a predetermined period.

According to the above-described configuration, a pulse wave signal having a great amplitude can be efficiently acquired at the time of blood pressure measurement by the depressurization measurement method.

In another example of the present disclosure, the blood pressure measurement unit specifies a cuff pressure at a timing at which the amplitude of the pulse wave signal is maximum in the pressurization process, and sets, as a predetermined period, a period from a timing at which the cuff pressure during the depressurization process reaches the specified cuff pressure until after elapse of a specified time.

According to the above-described configuration, a pulse wave signal having a great amplitude can be efficiently acquired at the time of blood pressure measurement by the depressurization measurement method.

In another example of the present disclosure, the blood pressure measurement unit sets the depressurization speed in the predetermined period to zero. According to the above-described configuration, more pulse wave signals having great amplitudes can be acquired at the time of blood pressure measurement by the depressurization measurement method.

In another example of the present disclosure, a method for measuring a blood pressure includes measuring a blood pressure of a user based on a pulse wave signal in a pressurization process of pressurizing a cuff pressure indicating an inner pressure of a cuff worn on a part of the user to be measured. The measuring includes setting a pressurization speed in a predetermined period of the pressurization process to be slower than a pressurization speed in a period other than the predetermined period of the pressurization process. The predetermined period is set based on a timing at which the amplitude of the pulse wave signal during the pressurization process is the maximum or the timing at which the cuff pressure during the pressurization process is an average blood pressure of the user. The method for measuring the blood pressure further includes determining an arrhythmia of the user based on a pulse wave signal in the pressurization process.

According to the above-described configuration, an arrhythmia can be accurately determined by acquiring many pulse wave signals having great amplitudes at the time of blood pressure measurement by the pressurization measurement method.

In another example of the present disclosure, a method for measuring a blood pressure includes measuring, after a pressurization process of pressurizing a cuff pressure indicating an inner pressure of a cuff worn on a part of the user to be measured up to a pressure greater than a specified pressure, a blood pressure of the user based on a pulse wave signal in a depressurization process of depressurizing the cuff pressure. The measuring includes setting a depressurization speed in a predetermined period of the depressurization process to be slower than a depressurization speed in a period other than the predetermined period of the depressurization process. The predetermined period is set based on a timing at which the amplitude of the pulse wave signal is maximum in the depressurization process or the timing at which the cuff pressure during the depressurization process is an average blood pressure of the user. The method for measuring the blood pressure further includes determining an arrhythmia of the user based on a pulse wave signal in the depressurization process.

According to the above-described configuration, an arrhythmia can be accurately determined by acquiring many pulse wave signals having great amplitudes at the time of blood pressure measurement by the depressurization measurement method.

### Advantageous Effects of Invention

According to the present disclosure, an arrhythmia can be accurately determined by acquiring many pulse wave signals having great amplitudes at the time of blood pressure measurement.

### BRIEF DESCRIPTION OF DRAWINGS

FIG. 1 is a diagram illustrating a sphygmomanometer according to an embodiment.

FIG. 2 is a block diagram illustrating an example of a hardware configuration of a sphygmomanometer.

FIG. 3 is a block diagram illustrating a functional configuration of the sphygmomanometer.

FIG. 4 is a diagram illustrating a correspondence relationship between a pulse wave signal and a cuff pressure at the time of blood pressure measurement (pressurization measurement method) in a normal mode.

FIG. 5 is a flowchart illustrating an example of a blood pressure measurement process (pressurization measurement method) in the normal mode.

FIG. 6 is a diagram illustrating a correspondence relationship between a pulse wave signal and a cuff pressure at the time of blood pressure measurement (depressurization measurement method) in the normal mode.

FIG. 7 is a flowchart illustrating a blood pressure measurement process (depressurization measurement method) in the normal mode.

FIG. 8 is a diagram illustrating a correspondence relationship between a pulse wave signal and a cuff pressure at the time of blood pressure measurement (pressurization measurement method) in an arrhythmia determination mode.

FIG. 9 is a flowchart illustrating a blood pressure measurement process (pressurization measurement method) in the arrhythmia determination mode.

FIG. 10 is a diagram illustrating a correspondence relationship between a pulse wave signal and a cuff pressure at the time of blood pressure measurement (depressurization measurement method) in the arrhythmia determination mode.

FIG. 11 is a flowchart illustrating a blood pressure measurement process (depressurization measurement method) in the arrhythmia determination mode.

FIG. 12 is a flowchart illustrating a first modified example of a blood pressure measurement process (pressurization measurement method) in the arrhythmia determination mode.

FIG. 13 is a flowchart illustrating the first modified example of a blood pressure measurement process (depressurization measurement method) in the arrhythmia determination mode.

FIG. 14 is a flowchart illustrating a second modified example of a blood pressure measurement process (pressurization measurement method) in the arrhythmia determination mode.

FIG. 15 is a flowchart illustrating the second modified example of a blood pressure measurement process (depressurization measurement method) in the arrhythmia determination mode.

FIG. 16 is a flowchart illustrating a third modified example of a blood pressure measurement process (depressurization measurement method) in the arrhythmia determination mode.

FIG. 17 is a flowchart illustrating a fourth modified example of a blood pressure measurement process (depressurization measurement method) in the arrhythmia determination mode.

FIG. 18 is a diagram illustrating a correspondence relationship between a pulse wave signal and a cuff pressure at the time of blood pressure measurement (pressurization measurement method) in the arrhythmia determination mode according to another embodiment.

FIG. 19 is a diagram illustrating a correspondence relationship between a pulse wave signal and a cuff pressure at the time of blood pressure measurement (depressurization measurement method) in the arrhythmia determination mode according to another embodiment.

FIG. 20 is a flowchart illustrating a blood pressure measurement process (pressurization measurement method) in the arrhythmia determination mode according to another embodiment.

FIG. 21 is a flowchart illustrating a blood pressure measurement process (depressurization measurement method) in the arrhythmia determination mode according to another embodiment.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### Application Example

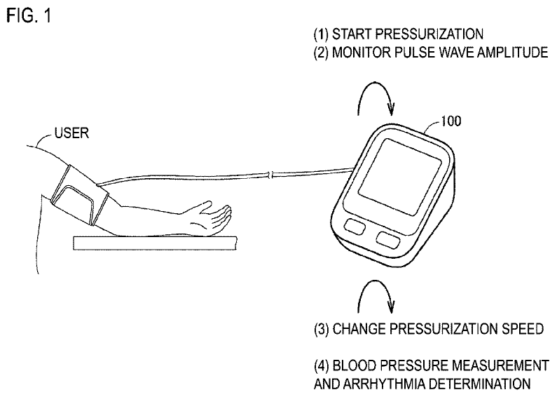

An application example of the present invention will be described with reference to FIG. 1. FIG. 1 is a diagram illustrating a sphygmomanometer 100 according to the present embodiment.

Referring to FIG. 1, the sphygmomanometer 100 is an upper-arm sphygmomanometer that measures a blood pressure by compressing a part of the user to be measured (i.e., the subject) with a cuff. The sphygmomanometer 100 executes blood pressure measurement through the oscillometric method. The sphygmomanometer 100 includes a main body and a cuff (arm band) as main components. The sphygmomanometer 100 may be a wrist-type sphygmomanometer in which a main body and a cuff (arm band) are integrated. Hereinafter, the processing contents will be described with reference to FIG. 1.

In FIG. 1, a scene in which a user measures his/her blood pressure using the sphygmomanometer 100 is assumed. The sphygmomanometer 100 measures the blood pressure of the user through a pressurization measurement method of measuring the blood pressure in a pressurization process of the cuff pressure indicating the inner pressure of the cuff worn on a part of the user to be measured (e.g., an arm).

The sphygmomanometer 100 starts pressurization of the cuff in response to a blood pressure measurement instruction of the user (corresponding to (1) in FIG. 1). It is assumed that the pressurization speed of the cuff pressure is set to a speed Va.

The sphygmomanometer 100 monitors the amplitude of the pulse wave signal (pulse wave amplitude) in the pressurization process of the cuff pressure (corresponding to (2) in FIG. 1). The pulse wave amplitude gradually increases as the cuff pressure increases, has reached the maximum value, and then gradually decreases. As described above, it is known that the pulse wave amplitude changes in a mountain shape in the pressurization process.

Subsequently, the sphygmomanometer 100 changes the pressurization speed based on the monitoring result of the pulse wave amplitude (corresponding to (3) in FIG. 1). For example, when determining that the pulse wave amplitude is the maximum, the sphygmomanometer 100 changes the pressurization speed to a speed Vb slower than the speed Va. The sphygmomanometer 100 continues to pressurize the cuff pressure at the speed Vb until a certain time elapses from the determination time point. After elapse of a certain time, the sphygmomanometer 100 returns the pressurization speed to the speed Va and continues pressurization of the cuff pressure.

The changing process of the pressurization speed is a process for acquiring a sufficient number of pulse wave signals for accurately detecting arrhythmia (e.g., atrial fibrillation) at the time of blood pressure measurement. Typically, the sphygmomanometer 100 determines the presence or absence of occurrence of arrhythmia based on the interval (pulse wave interval) of the pulse wave signals acquired at the time of blood pressure measurement. Therefore, in order to execute the determination with high accuracy, it is preferable to acquire more pulse wave signals having a great amplitude. Therefore, the sphygmomanometer 100 acquires many pulse wave signals having a great amplitude by lowering the pressurization speed in a period in which a great pulse wave amplitude is obtained.

Then, the sphygmomanometer 100 calculates the blood pressure value of the user and determines the presence or absence of occurrence of arrhythmia based on the pulse wave signal obtained in the pressurization process (corresponding to (4) in FIG. 1). In this case, the sphygmomanometer 100 displays the blood pressure value of the user and the determination result of the arrhythmia on the display.

According to the above application example, in the pressurization process, the cuff pressure is pressurized at a slow pressurization speed in a period in which the pulse wave amplitude is great, and the cuff pressure is pressurized at a normal pressurization speed in other periods. As a result, more pulse wave signals having a great amplitude can be more efficiently acquired, and as a result, the arrhythmia can be accurately determined.

In addition, it is known that the cuff pressure at the timing at which the pulse wave amplitude is the maximum is around the average blood pressure of the user. Thus, it can be said that the period in which the cuff pressure is around the average blood pressure is a period in which the pulse wave amplitude is great. Therefore, the sphygmomanometer 100 may change the pressurization speed to the speed Vb slower than the speed Va when determining that the cuff pressure is the average blood pressure of the user. In this case, since the pressurization speed is slower in a period in which the cuff pressure is around the average blood pressure, many pulse wave signals having a great amplitude can be acquired.

### Configuration Example

### Hardware Configuration

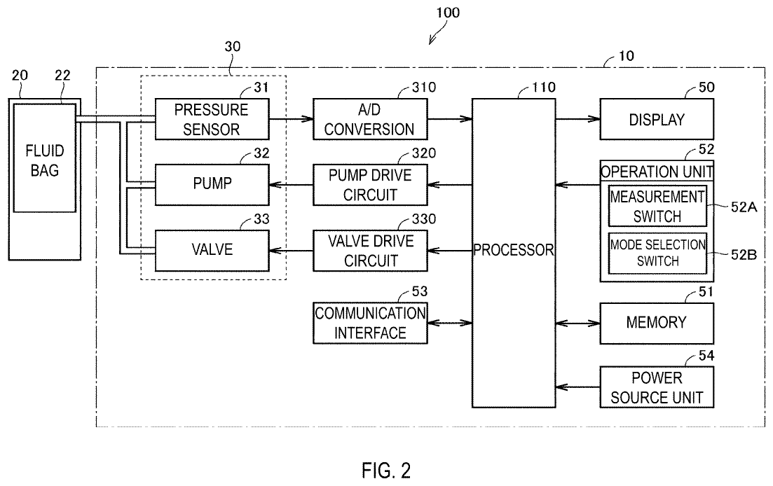

FIG. 2 is a block diagram illustrating an example of a hardware configuration of a sphygmomanometer 100. Referring to FIG. 2, the sphygmomanometer 100 includes a main body 10 and a cuff 20 as main components. The cuff 20 interiorly includes a fluid bag 22. The main body 10 includes a processor 110, an air-system component 30 for blood pressure measurement, an A/D conversion circuit 310, a pump drive circuit 320, a valve drive circuit 330, a display 50, a memory 51, an operation unit 52, a communication interface 53, and a power source unit 54.

The processor 110 may typically be an arithmetic processing unit such as a Central Processing Unit (CPU) or a Multi Processing Unit (MPU). The processor 110 reads and executes the program stored in the memory 51 to implement each process (step) of the sphygmomanometer 100 described later. For example, the processor 110 performs control to drive the pump 32 and the valve 33 in response to an operation signal from the operation unit 52. In addition, the processor 110 calculates a blood pressure value using an algorithm for blood pressure calculation by an oscillometric method and displays the blood pressure value on the display 50.

The memory 51 is achieved by a Random Access Memory (RAM), a Read-Only Memory (ROM), a flash memory, or the like. The memory 51 stores a program for controlling the sphygmomanometer 100, data used for controlling the sphygmomanometer 100, setting data for setting various functions of the sphygmomanometer 100, data of a measurement result of a blood pressure value, a pulse rate, a pulse wave interval, and the like. Furthermore, the memory 51 is used as working memory and the like for when executing a program.

The air-system component 30 supplies or discharges air to or from the fluid bag 22 interiorly contained in the cuff 20 through an air line. The air-system component 30 includes a pressure sensor 31 for detecting a pressure in the fluid bag 22, and a pump 32 and a valve 33 serving as an expanding/contracting mechanism section for expanding/contracting the fluid bag 22.

The pressure sensor 31 detects a pressure (cuff pressure) in the fluid bag 22 and outputs a signal (cuff pressure signal) corresponding to the detected pressure to the A/D conversion circuit 310. The pressure sensor 31 is, for example, a piezo-resistive pressure sensor connected to the pump 32, the valve 33, and the fluid bag 22 interiorly contained in the cuff 20 via the air line. The pump 32 supplies air as a fluid to the fluid bag 22 through an air line in order to pressurize the cuff pressure. The valve 33 is opened and closed to control the cuff pressure by discharging air in the fluid bag 22 through the air line or enclosing air in the fluid bag 22.

The A/D conversion circuit 310 converts an output value of the pressure sensor 31 (e.g., a voltage value corresponding to a change in electric resistance due to a piezo-resistive effect) from an analog signal to a digital signal and outputs the converted signal to the processor 110. The processor 110 acquires a signal representing the cuff pressure according to the output value of the A/D conversion circuit 310. The pump drive circuit 320 controls driving of the pump 32 based on a control signal provided from the processor 110. The valve drive circuit 330 controls opening and closing of the valve 33 based on a control signal provided from the processor 110.

The processor 110 executes blood pressure measurement by a pressurization measurement method of measuring the blood pressure of the user based on a pulse wave signal in a pressurization process of pressurizing the cuff pressure or a depressurization measurement method of measuring the blood pressure of the user based on a pulse wave signal in a depressurization process of depressurizing the cuff pressure after the pressurization process of pressurizing the cuff pressure to a pressure greater than a specified pressure (e.g., “estimated systolic blood pressure” described later).

For example, at the time of measurement by the depressurization measurement method, the following operation is generally performed. A cuff is wound around a part of the user to be measured (wrist, arm, etc.) in advance, and at the time of measurement, the pump 32 and the valve 33 are controlled to pressurize the cuff pressure to be higher than the estimated systolic blood pressure, and then gradually depressurizes the cuff pressure. In the process of depressurization, the cuff pressure is detected by the pressure sensor 31, and the variation of arterial volume occurring in the artery of the part to be measured is taken out as a pulse wave signal. The maximal blood pressure (systolic blood pressure) and the minimal blood pressure (diastolic blood pressure) are calculated based on the change (mainly, a rise and fall) in amplitude of the pulse wave signal involved in the change in the cuff pressure at the time.

The operation unit 52 inputs an operation signal corresponding to an instruction by a user to the processor. The operation unit 52 includes a measurement switch 52A for receiving a blood pressure measurement instruction from the user, and a mode selection switch 52B for selecting a measuring mode.

When the measurement switch 52A is pressed, the part to be measured is temporarily compressed by the cuff 20, and the blood pressure measurement is executed through the oscillometric method. When the measurement switch 52A is pressed again during the blood pressure measurement, the blood pressure measurement is stopped.

Furthermore, when the mode selection switch 52B is pressed, the measuring mode is switched. For example, in a case where the mode selection switch 52B is pressed when the current measuring mode is set to the normal measuring mode (hereinafter also simply referred to as the “normal mode”), the measuring mode is switched to an arrhythmia determination mode.

The display 50 displays various kinds of information including a blood pressure measurement result and the like based on a control signal from the processor 110. The communication interface 53 exchanges various kinds of information with an external device. The power source unit 54 supplies power to the processor 110 and each piece of hardware.

### Functional Configuration

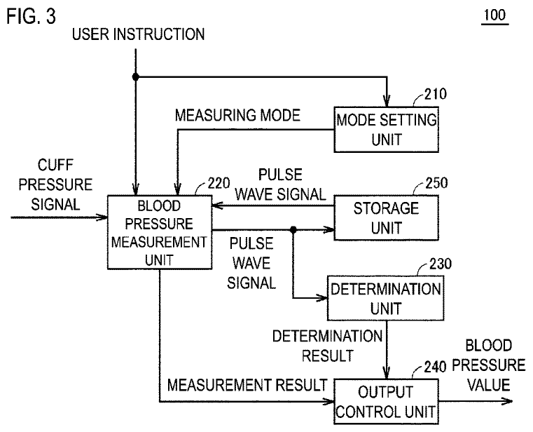

FIG. 3 is a block diagram illustrating the functional configuration of the sphygmomanometer 100. Referring to FIG. 3, the sphygmomanometer 100 includes a mode setting unit 210, a blood pressure measurement unit 220, a determination unit 230, and an output control unit 240 as a main functional configuration. Each of these functions is realized, for example, by the processor 110 of the sphygmomanometer 100 executing a program stored in the memory 51. Note that some or all of these functions may be configured to be realized by hardware. The sphygmomanometer 100 further includes a storage unit 250. The storage unit 250 is realized by the memory 51.

The mode setting unit 210 sets one of an arrhythmia determination mode in which determination of presence or absence of arrhythmia of the user is executed and a normal mode in which determination of arrhythmia is not executed. Typically, the mode setting unit 210 sets either the arrhythmia determination mode or the normal mode in response to a mode selection instruction from the user via the operation unit 52 (e.g., the mode selection switch 52B).

Note that the mode setting unit 210 may be configured to automatically set either mode according to a schedule defined in advance. For example, when the blood pressure measurement is started (e.g., blood pressure measurement is started by pressing the measurement switch 52A) in the time zone H of the day, the arrhythmia determination mode is automatically set. On the other hand, when the blood pressure measurement is executed in a time zone other than the time zone H in the day, the normal mode is automatically set.

The blood pressure measurement unit 220 controls the cuff pressure in response to a measurement start instruction from the user via the operation unit 52 (e.g., the measurement switch 52A). Specifically, the blood pressure measurement unit 220 drives the pump 32 via the pump drive circuit 320 and performs a control for driving the valve 33 via the valve drive circuit 330. The valve 33 is opened and closed to discharge or enclose the air in the fluid bag 22 and control the cuff pressure.

The blood pressure measurement unit 220 receives the cuff pressure signal detected by the pressure sensor 31 and takes out a pulse wave signal representing the pulse wave of the part to be measured superimposed on the cuff pressure signal. That is, the blood pressure measurement unit 220 detects a pulse wave, which is a pressure component superimposed on the cuff pressure signal in synchronization with the pulsation of the heart of the user, from the cuff pressure signal.

The blood pressure measurement unit 220 calculates blood pressure information of the user based on the cuff pressure signal and the pulse wave signal superimposed on the cuff pressure signal. Specifically, the blood pressure measurement unit 220 measures the blood pressure of the user by a pressurization measurement method or a depressurization measurement method according to the oscillometric method. Typically, the blood pressure measurement unit 220 calculates systolic blood pressure, diastolic blood pressure, pulse rate, pulse pressure, and the like. The storage unit 250 stores information obtained at the time of blood pressure measurement (e.g., cuff pressure, pulse wave signal, systolic blood pressure, diastolic blood pressure, pulse rate, pulse pressure, etc.).

Here, assume that the measuring mode is set to the normal mode by the mode setting unit 210. In this case, when executing the blood pressure measurement by the pressurization measurement method, the blood pressure measurement unit 220 makes the pressurization speed in the pressurization process constant. Furthermore, when executing the blood pressure measurement by the depressurization measurement method, the blood pressure measurement unit 220 makes the depressurization speed in the depressurization process constant.

Assume that the measuring mode is set to the arrhythmia determination mode by the mode setting unit 210. First, a case where the blood pressure measurement unit 220 executes blood pressure measurement by a pressurization measurement method will be described.

When executing the blood pressure measurement by the pressurization measurement method, the blood pressure measurement unit 220 makes the pressurization speed in the period Tb of the pressurization process slower than the pressurization speed in another period Ta other than the period Tb in the pressurization process. The period Tb is set based on a timing at which the amplitude of the pulse wave signal during the pressurization process is the maximum or the timing at which the cuff pressure during the pressurization process is an average blood pressure of the user.

In a certain aspect, the blood pressure measurement unit 220 sets, as the period Tb, a period from a timing at which the amplitude of the pulse wave signal during the pressurization process is the maximum until after elapse of a specified time.

In another aspect, the blood pressure measurement unit 220 sets the period Tb based on the related information stored in storage unit 250. Specifically, the storage unit 250 stores related information in which the cuff pressure and the pulse wave signal obtained at the time of past blood pressure measurement of the user are associated with each other. For example, the related information includes information indicating a correspondence relationship between the cuff pressure and the pulse wave signal in the pressurization process or the depressurization process when the blood pressure of the user is measured.

The blood pressure measurement unit 220 extracts the cuff pressure at a timing at which the amplitude of the pulse wave signal in the pressurization process is maximum at the time of past blood pressure measurement of the user based on the related information. The blood pressure measurement unit 220 sets, as the period Tb, a period from a timing at which the cuff pressure has reached the extracted cuff pressure in the pressurization process at the time of current blood pressure measurement of the user until after elapse of a specified time.

In still another aspect, the blood pressure measurement unit 220 sets the period Tb based on the average blood pressure of the user. Specifically, the storage unit 250 stores the systolic blood pressure and the diastolic blood pressure of the user obtained at the time of past blood pressure measurement of the user. The blood pressure measurement unit 220 calculates the average blood pressure based on the systolic blood pressure and the diastolic blood pressure stored in the storage unit 250. For example, the blood pressure measurement unit 220 calculates the average blood pressure based on the relational expression “average blood pressure=diastolic blood pressure+(systolic blood pressure−diastolic blood pressure)÷3”. The blood pressure measurement unit 220 sets, as the period Tb, a period from a timing at which the cuff pressure in the pressurization process at the time of current blood pressure measurement of the user has reached the average blood pressure until after elapse of a specified time.

Note that the blood pressure measurement unit 220 may set the pressurization speed to zero in the period Tb of the pressurization process. That is, the blood pressure measurement unit 220 may maintain the cuff pressure constant by stopping the pressurization of the cuff pressure in the period Tb.

Next, a case where the arrhythmia determination mode is set and the blood pressure measurement unit 220 executes the blood pressure measurement by the depressurization measurement method will be described. In this case, the blood pressure measurement unit 220 makes the depressurization speed in the period Tg of the depressurization process slower than the depressurization speed in another period Tf other than the period Tg in the depressurization process. The period Tg is set based on a timing at which the amplitude of the pulse wave signal during the depressurization process is the maximum or the timing at which the cuff pressure during the depressurization process is an average blood pressure of the user.

In a certain aspect, the blood pressure measurement unit 220 sets, as the period Tg, a period from a timing at which the amplitude of the pulse wave signal during the depressurization process is the maximum until after elapse of a specified time.

In another aspect, the blood pressure measurement unit 220 extracts the cuff pressure at a timing at which the amplitude of the pulse wave signal in the depressurization process is maximum at the time of past blood pressure measurement of the user based on the related information stored in the storage unit 250. The blood pressure measurement unit 220 sets, as the period Tg, a period from a timing at which the cuff pressure in the depressurization process at the time of current blood pressure measurement of the user has reached the extracted cuff pressure until after elapse of the specified time.

In still another aspect, the blood pressure measurement unit 220 sets the period Tg based on the average blood pressure of the user. Specifically, the blood pressure measurement unit 220 calculates the average blood pressure based on the systolic blood pressure and the diastolic blood pressure stored in the storage unit 250. The blood pressure measurement unit 220 sets, as the period Tg, a period from a timing at which the cuff pressure in the depressurization process at the time of current blood pressure measurement of the user has reached the average blood pressure until after elapse of a specified time.

In still another aspect, in the pressurization process of the depressurization measurement method, the blood pressure measurement unit 220 estimates the systolic blood pressure and the diastolic blood pressure of the user, and estimates the average blood pressure (i.e., calculates the estimated average blood pressure) based on the estimated systolic blood pressure (hereinafter, also referred to as “estimated systolic blood pressure”) and diastolic blood pressure (hereinafter, also referred to as “estimated diastolic blood pressure”). The systolic blood pressure and the diastolic blood pressure are estimated by a known method. For example, the blood pressure measurement unit 220 estimates the systolic blood pressure and the diastolic blood pressure from a pulse wave envelope indicating the pattern of the amplitude change of the pulse wave signal that changes in the process of pressurizing the cuff pressure. The blood pressure measurement unit 220 calculates the estimated average blood pressure based on the relational expression “estimated average blood pressure=estimated diastolic blood pressure+(estimated systolic blood pressure−estimated diastolic blood pressure)÷3”. The blood pressure measurement unit 220 sets, as the period Tg, a period from a timing at which the cuff pressure in the depressurization process has reached the estimated average blood pressure until after elapse of a specified time.

In still another aspect, the blood pressure measurement unit 220 specifies the cuff pressure at a timing at which the amplitude of the pulse wave signal during the pressurization process is the maximum of the depressurization measurement method. The blood pressure measurement unit 220 sets, as the period Tg, a period from a timing at which the cuff pressure in the depressurization process has reached the specified cuff pressure until after elapse of a specified time.

Note that the blood pressure measurement unit 220 may set the depressurization speed in the period Tg of the depressurization process to zero. That is, the blood pressure measurement unit 220 may maintain the cuff pressure constant by stopping the depressurization of the cuff pressure in the period Tg.

The determination unit 230 executes the arrhythmia determination when the arrhythmia determination mode is set. When the blood pressure measurement unit 220 executes blood pressure measurement by the pressurization measurement method, the determination unit 230 determines whether or not arrhythmia of the user has occurred based on the pulse wave signal in the pressurization process. When the blood pressure measurement unit 220 executes the blood pressure measurement by the depressurization measurement method, the determination unit 230 determines whether or not arrhythmia of the user has occurred based on the pulse wave signal in the depressurization process. A known method is used for a method for determining arrhythmia. For example, the determination unit 230 determines the presence or absence of occurrence of arrhythmia based on the occurrence intervals (i.e., pulse wave interval) of the plurality of pulse waves acquired from the pulse wave signal.

The output control unit 240 displays the measurement result of the blood pressure measurement unit 220, the determination result of the determination unit 230, and the like on the display 50. Note that the output control unit 240 may transmit the measurement result and the determination result to an external device via the communication interface 53, or may be configured to output a voice via a speaker (not illustrated).

### Process at Time of Normal Mode

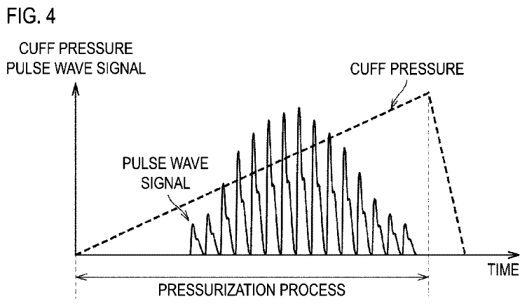

FIG. 4 is a diagram illustrating a correspondence relationship between a pulse wave signal and a cuff pressure at the time of blood pressure measurement (pressurization measurement method) in a normal mode. Referring to FIG. 4, the sphygmomanometer 100 starts a pressurization process and pressurizes the cuff at a constant speed (e.g., speed Va). In the pressurization process, the pulse wave amplitude gradually increases as the cuff pressure increases and gradually decreases after reaching the maximum point. When the blood pressure measurement is completed, the sphygmomanometer 100 stops the pump 32, ends the pressurization process, and opens the valve 33. As a result, the cuff pressure rapidly decreases.

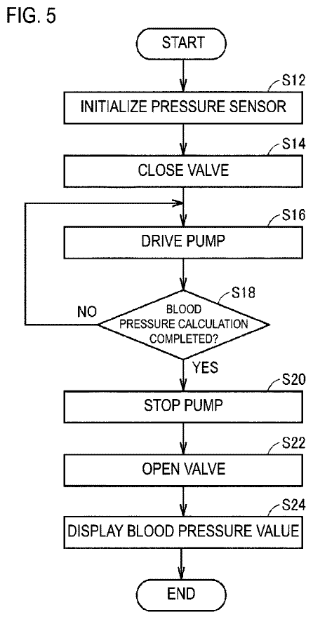

FIG. 5 is a flowchart illustrating an example of a blood pressure measurement process (pressurization measurement method) in the normal mode.

Referring to FIG. 5, the processor 110 of the sphygmomanometer 100 initializes the pressure sensor 31 (step S12). Specifically, the processor 110 initializes the processing memory area, and performs 0 mmHg adjustment (setting the atmospheric pressure to 0 mmHg) of the pressure sensor 31 in a state where the pump 32 is turned off (stopped) and the valve 33 is opened.

Next, the processor 110 closes the valve 33 via the valve drive circuit 330 (step S14), and drives the pump 32 via the pump drive circuit 320 to start pressurization of the cuff 20 (fluid bag 22) (step S16). At this time, the processor 110 controls the pressurization speed of the cuff pressure, which is the pressure in the fluid bag 22, based on the output of the pressure sensor 31 while supplying air from the pump 32 to the fluid bag 22 through the air line. This starts the pressurization process. The processor 110 controls the pressurization speed to a constant speed (e.g., speed Va).

Next, the processor 110 extracts a pulse wave signal from the cuff pressure signal detected by the pressure sensor 31, attempts to calculate the systolic blood pressure and the diastolic blood pressure based on the pulse wave signal, and determines whether or not the blood pressure calculation has been completed (step S18).

When the blood pressure calculation cannot be completed yet due to lack of data (NO in step S18), the processor 110 repeats the processes of steps S16 and S18 as long as the cuff pressure has not reached an upper limit pressure defined in advance (e.g., 300 mmHg). When the blood pressure calculation is completed (YES in step S18), the processor 110 stops the pump 32 (step S20), opens the valve 33 (step S22), and performs a control to exhaust the air in the cuff 20. The processor 110 displays the blood pressure value measured by the blood pressure measurement on the display 50 (step S24).

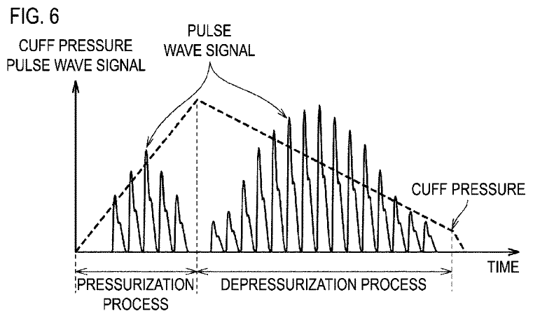

FIG. 6 is a diagram illustrating a correspondence relationship between a pulse wave signal and a cuff pressure at the time of blood pressure measurement (depressurization measurement method) in the normal mode. Referring to FIG. 6, the sphygmomanometer 100 starts the pressurization process and pressurizes the cuff pressure until the cuff pressure has reached a specified pressure (e.g., the estimated systolic blood pressure). At this time, as the cuff pressure increases, the pulse wave amplitude increases and then decreases.

When the cuff pressure has reached the specified pressure, the sphygmomanometer 100 shifts from the pressurization process to the depressurization process to depressurize the cuff pressure. In the depressurization process, the pulse wave amplitude gradually increases as the cuff pressure decreases, and after reaching a maximum point, gradually decreases. When the blood pressure measurement is completed, the sphygmomanometer 100 ends the depressurization process and fully opens the valve 33.

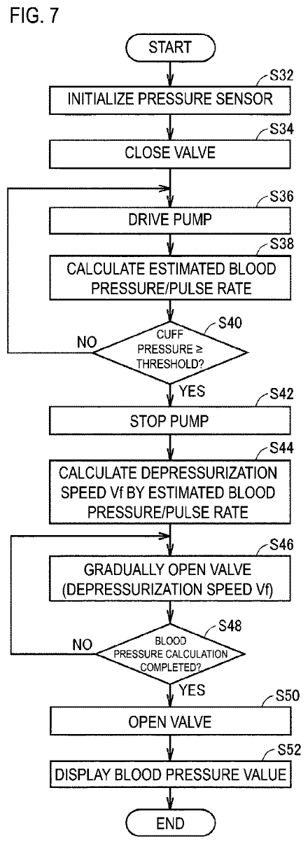

FIG. 7 is a flowchart illustrating a blood pressure measurement process (depressurization measurement method) in the normal mode. Referring to FIG. 7, since the processes of steps S32 to S36 are similar to the processes of steps S12 to S16 of FIG. 5, the detailed description thereof will not be repeated.

The processor 110 calculates the estimated systolic blood pressure, the estimated diastolic blood pressure, and the pulse rate based on the pulse wave signal obtained in the pressurization process (step S38). Subsequently, the processor 110 determines whether or not the cuff pressure has reached greater than or equal to a threshold Th (step S40). Typically, the threshold Th is set to a value higher than the estimated systolic blood pressure by a fixed value (e.g., 40 mmHg).

When the cuff pressure is less than the threshold Th (NO in step S40), the processor 110 returns to step S36. When the cuff pressure is greater than or equal to the threshold Th (YES in step S40), the processor 110 stops the pump 32 (step S42). The processor 110 calculates a depressurization speed (e.g., speed Vf) based on the estimated pulse pressure which is the difference between the estimated systolic blood pressure and the estimated diastolic blood pressure and the pulse rate (step S44). Typically, the processor 110 sets the depressurization speed so that the pulse rate generated between the estimated pulse pressures is greater than or equal to a predetermined pulse rate.

The processor 110 controls the valve 33 to gradually open at the speed Vf (step S46). As a result, the pressurization process shifts to the depressurization process, and the cuff pressure is gradually depressurized.

In this depressurization process, the processor 110 extracts a pulse wave signal from the cuff pressure signal detected by the pressure sensor 31, attempts to calculate the systolic blood pressure and the diastolic blood pressure based on the pulse wave signal, and determines whether or not the blood pressure calculation is completed (step S48). When the blood pressure calculation is not completed (NO in step S48), the processor 110 repeats the processes of steps S46 and S48. When the blood pressure calculation is completed (YES in step S48), the processor 110 performs a control to fully open the valve 33 (step S50) and rapidly exhaust the air in the cuff 20. The processor 110 displays the blood pressure value measured by the blood pressure measurement on the display 50 (step S52).

### Process at Time of Arrhythmia Determination Mode

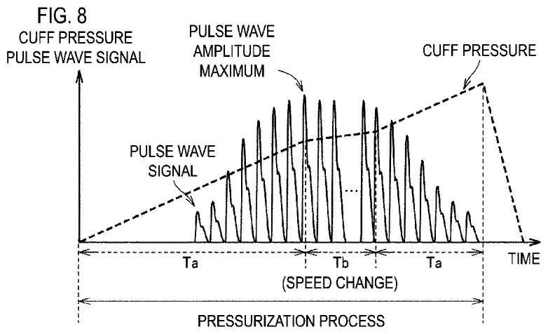

FIG. 8 is a diagram illustrating a correspondence relationship between a pulse wave signal and a cuff pressure at the time of blood pressure measurement (pressurization measurement method) in the arrhythmia determination mode. Referring to FIG. 8, the sphygmomanometer 100 starts a pressurization process and pressurizes the cuff pressure at a constant pressurization speed (e.g., speed Va). In the pressurization process, the pulse wave amplitude gradually increases as the cuff pressure increases and reaches a maximum point.

When determining that the pulse wave amplitude has reached a maximum, the sphygmomanometer 100 changes the pressurization speed from the speed Va to the speed Vb (however, the speed Vb is slower than the speed Va). The speed change period in which the cuff pressure is pressurized at the speed Vb is a period from the determination time point until after elapse of a specified time. The speed change period corresponds to the period Tb described above, and another period other than the speed change period in the pressurization process corresponds to the period Ta described above.

When the period Tb ends, the sphygmomanometer 100 returns the pressurization speed from the speed Vb to the speed Va and continues the pressurization of the cuff. When the blood pressure measurement is completed, the pump 32 is stopped to end the pressurization process, and the valve 33 is opened. As a result, the cuff pressure rapidly decreases.

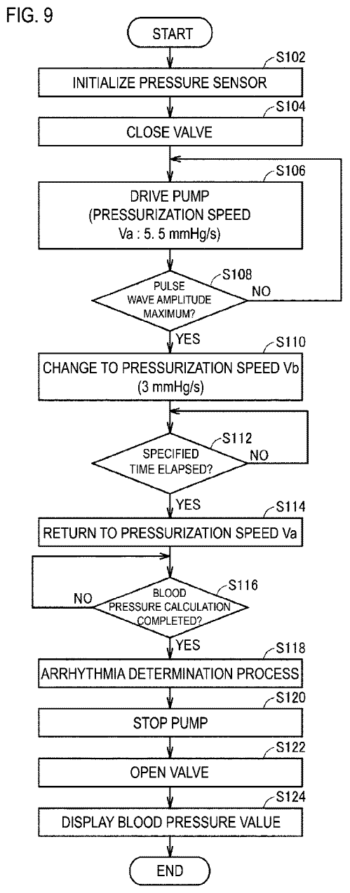

FIG. 9 is a flowchart illustrating a blood pressure measurement process (pressurization measurement method) in the arrhythmia determination mode.

Referring to FIG. 9, since the processes of steps S102 to S106 are similar to the processes of steps S12 to S16 of FIG. 5, the detailed description thereof will not be repeated. Note that in step S106, the processor 110 is assumed to pressurize the cuff pressure at the speed Va (e.g., 5.5 mmHg/s). This starts the pressurization process.

The processor 110 determines whether or not the pulse wave amplitude has reached a maximum in the pressurization process (step S108). For example, it is assumed that the change rate of the pulse wave amplitude in a case where the pulse wave amplitude is increasing is positive, and the change rate of the pulse wave amplitude in a case where the pulse wave amplitude is decreasing is negative. In this case, the processor 110 determines that the pulse wave amplitude is a maximum at a timing at which the change rate of the pulse wave amplitude changed from positive to negative.

When the pulse wave amplitude is not maximum (NO in step S108), the processor 110 executes step S106 to continue the pressurization of the cuff pressure at the speed Va. When the pulse wave amplitude is a maximum (YES in step S108), the processor 110 changes the pressurization speed from the speed Va to the speed Vb (e.g., 3 mmHg/s) (step S110).

The processor 110 determines whether or not a specified time has elapsed since the pressurization speed changed to the speed Vb (step S112). When the specified time has not elapsed (NO in step S112), the processor 110 repeats the process in step S112. When the specified time has elapsed (YES in step S112), the processor 110 returns the pressurization speed from the speed Vb to the speed Va (step S114).

The processor 110 attempts to calculate the systolic blood pressure and the diastolic blood pressure based on the pulse wave signal in the pressurization process, and determines whether or not the blood pressure calculation is completed (step S116). When the blood pressure calculation cannot be completed yet due to lack of data (NO in step S116), the processor 110 repeats the process of step S116 as long as the cuff pressure has not reached an upper limit pressure defined in advance (e.g., 300 mmHg). That is, the processor 110 attempts to calculate the blood pressure while continuing the pressurization of the cuff pressure at the speed Va.

When the blood pressure calculation is completed (YES in step S116), the processor 110 executes the arrhythmia determination process (step S118). Specifically, the processor 110 determines the presence or absence of occurrence of arrhythmia of the user based on the pulse wave interval acquired from the pulse wave signals in the pressurization process. Subsequently, the processor 110 executes the processes of steps S120 to S124. Since the processes in steps S120 to S124 are similar to the processes in steps S20 to S24 in FIG. 5, the detailed description thereof will not be repeated. Note that in step S124, the processor 110 displays the determination result of the arrhythmia together with the blood pressure value on the display 50.

In the example of FIG. 9, the speed change period (i.e., the period Tb) in which the cuff pressure is pressurized at the speed Vb is a period from a timing at which the pulse wave amplitude is the maximum until after elapse of a specified time.

The timing of changing the pressurization speed from the speed Va to the speed Vb (i.e., the start timing of the period Tb) may be set to a timing before the timing at which the pulse wave amplitude is the maximum. In this case, the start timing of the period Tb may be set to a timing immediately before the change rate of the pulse wave amplitude changes from positive to negative (e.g., timing at which the positive change rate is less than the threshold).

Alternatively, the start timing of the period Tb may be set to a timing after the timing at which the pulse wave amplitude is the maximum. In this case, the start timing of the period Tb may be set after elapse of a predetermined time (e.g., one second) from a timing at which determination is made that the pulse wave amplitude has reached the maximum.

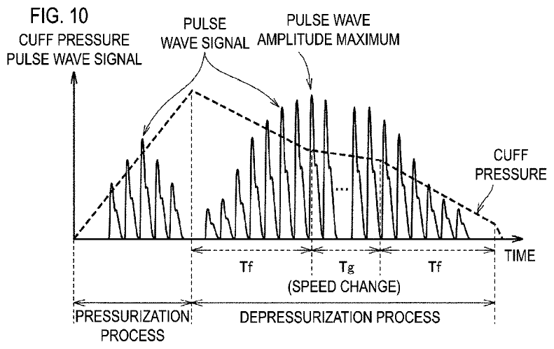

FIG. 10 is a diagram illustrating a correspondence relationship between a pulse wave signal and a cuff pressure at the time of blood pressure measurement (depressurization measurement method) in the arrhythmia determination mode. Referring to FIG. 10, the sphygmomanometer 100 starts the pressurization process and pressurizes the cuff pressure at a constant pressurization speed. When the cuff pressure has reached the specified pressure, the sphygmomanometer 100 shifts from the pressurization process to the depressurization process to depressurize the cuff pressure at the speed Vf. In the depressurization process, the pulse wave amplitude gradually increases and has reached a maximum point as the cuff pressure decreases.

When determining that the pulse wave amplitude has is the maximum, the sphygmomanometer 100 changes the depressurization speed from the speed Vf to the speed Vg (however, the speed Vg is sufficiently slower than the speed Vf). The speed change period during which the cuff pressure is depressurized at the speed Vf is a period from the determination time point until after elapse of a specified time. The speed change period corresponds to the period Tg described above, and other periods in the depressurization process other than the speed change period correspond to the period Tf described above.

When the period Tg ends, the sphygmomanometer 100 returns the depressurization speed from the speed Vg to the speed Vf and continues the depressurization of the cuff. When the blood pressure measurement is completed, the sphygmomanometer 100 ends the depressurization process and fully opens the valve 33.

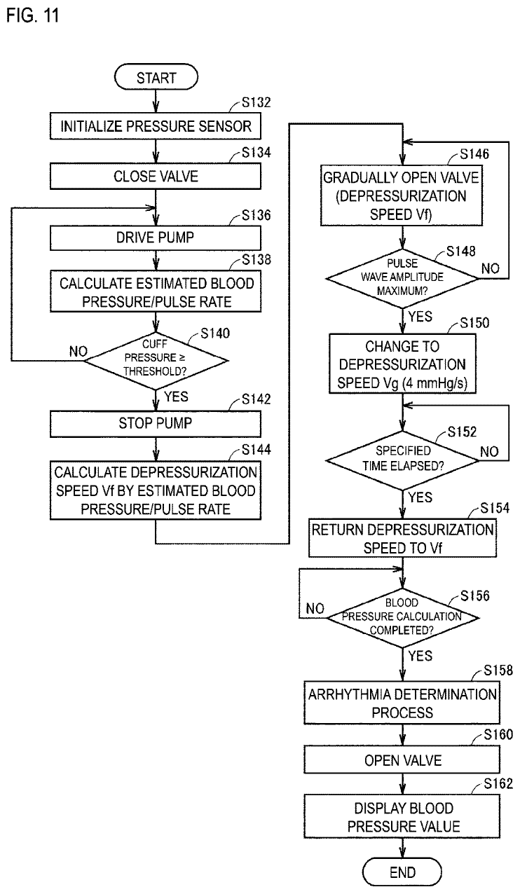

FIG. 11 is a flowchart illustrating a blood pressure measurement process (depressurization measurement method) in the arrhythmia determination mode.

Referring to FIG. 11, since the processes of steps S132 to S146 are similar to the processes of steps S32 to S46 of FIG. 7, the detailed description thereof will not be repeated. In step S146, the processor 110 depressurizes the cuff pressure at the speed Vf to start the depressurization process.

The processor 110 determines whether or not the pulse wave amplitude has reached the maximum in the depressurization process (step S148). When the pulse wave amplitude is not the maximum (NO in step S148), the processor 110 executes step S146 to continue the depressurization of the cuff pressure at the speed Vf. When the pulse wave amplitude is the maximum (YES in step S148), the processor 110 changes the depressurization speed from the speed Vf to the speed Vg (e.g., 4 mmHg/s) (step S150). The speed Vg is sufficiently slower than the speed Vf.

The processor 110 determines whether or not a specified time has elapsed since the depressurization speed changed to the speed Vg (step S152). When the specified time has not elapsed (NO in step S152), the processor 110 repeats the process in step S152. When the specified time has elapsed (YES in step S152), the processor 110 returns the depressurization speed from the speed Vg to the speed Vf (step S154).

The processor 110 attempts to calculate the systolic blood pressure and the diastolic blood pressure based on the pulse wave signal in the depressurization process, and determines whether or not the blood pressure calculation is completed (step S156). When the blood pressure calculation cannot be completed (NO in step S156), the processor 110 executes the process of step S156. That is, the processor 110 attempts to calculate the blood pressure while continuing the depressurization of the cuff pressure at the speed Vf.

When the blood pressure calculation is completed (YES in step S156), the processor 110 executes the arrhythmia determination process (step S158). Subsequently, the processor 110 fully opens the valve 33 (step S160), and displays the determination result of the arrhythmia together with the blood pressure value on the display 50 (step S162).

In the example of FIG. 11, the speed change period (i.e., the period Tg) in which the cuff pressure is depressurized at the speed Vg is a period from a timing at which the pulse wave amplitude is the maximum until after elapse of a specified time. The timing (i.e., the start timing of the period Tg) at which the depressurization speed is changed from the speed Vf to the speed Vg may be set to the timing before and after the timing at which the pulse wave amplitude is the maximum.

### First Modified Example

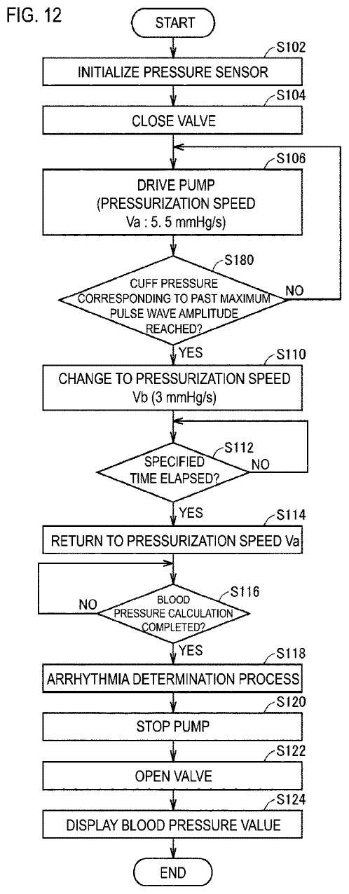

FIG. 12 is a flowchart illustrating a first modified example of a blood pressure measurement process (pressurization measurement method) in the arrhythmia determination mode.

Referring to FIG. 12, the processes in steps S102 to S106 and S110 to S124 are similar to the corresponding processes in FIG. 9, and thus the detailed description thereof will not be repeated.

After the process of step S106, the processor 110 determines whether or not the pressurized cuff pressure has reached the cuff pressure corresponding to the maximum value of the pulse wave amplitude at the time of past blood pressure measurement of the user (step S180). Specifically, the processor 110 reads, from the memory 51, related information indicating the relationship between the cuff pressure and the pulse wave amplitude at the time of past blood pressure measurement (e.g., at the time of previous measurement). Based on the related information, the processor 110 extracts the cuff pressure at a timing at which the pulse wave amplitude in the pressurization process is the maximum (i.e., the maximum pulse wave amplitude is obtained) at the time of previous measurement. The processor 110 determines whether or not the cuff pressure in the pressurization process (i.e., the cuff pressure currently being pressurized) at the time of current blood pressure measurement has reached the extracted cuff pressure.

When the cuff pressure being pressurized has not reached the extracted cuff pressure (NO in step S180), the processor 110 executes step S106. When the cuff pressure being pressurized has reached the extracted cuff pressure (YES in step S180), the processor 110 changes the pressurization speed from the speed Va to the speed Vb (e.g., 3 mmHg/s) (step S110).

In the example of FIG. 12, the speed change period (i.e., the period Tb) in which the cuff pressure is pressurized at the speed Vb is a period from the time point at which determination is made that the cuff pressure at the time of current measurement has reached the extracted cuff pressure until after elapse of a specified time. Note that in step S180 described above, the processor 110 may extract the cuff pressure at a timing at which the pulse wave amplitude smaller by a predetermined value (alternatively, greater by a predetermined value) than the maximum pulse wave amplitude in the pressurization process at the time of previous measurement is obtained based on the related information. However, the predetermined value is assumed to be a small value.

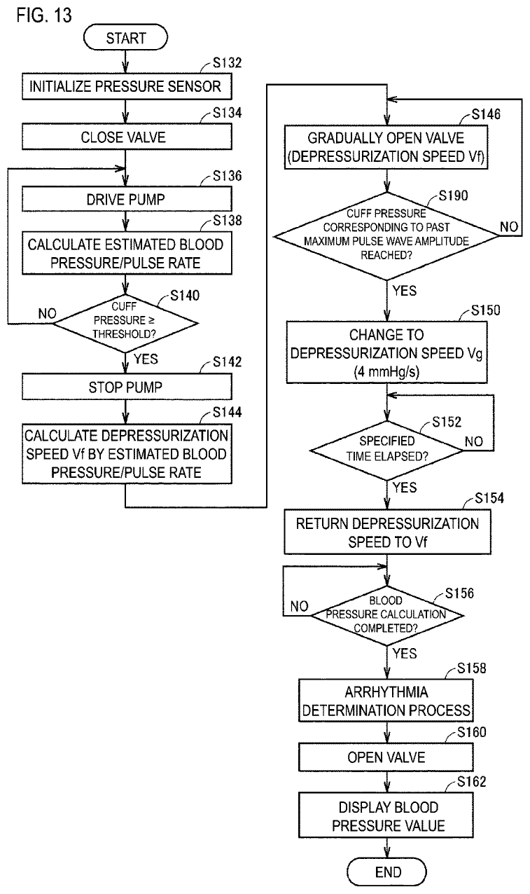

FIG. 13 is a flowchart illustrating a first modified example of a blood pressure measurement process (depressurization measurement method) in the arrhythmia determination mode.

Referring to FIG. 13, the processes in steps S132 to S146 and S150 to S162 are similar to the corresponding processes in FIG. 11, and thus, will not be described in detail.

After the process of step S146, the processor 110 determines whether or not the depressurized cuff pressure has reached the cuff pressure corresponding to the maximum value of the pulse wave amplitude at the time of past blood pressure measurement of the user (step S190). Specifically, the processor 110 reads, from the memory 51, related information indicating the relationship between the cuff pressure and the pulse wave amplitude at the time of past blood pressure measurement (e.g., at the time of previous measurement). Based on the related information, the processor 110 extracts the cuff pressure at a timing at which the pulse wave amplitude in the depressurization process is the maximum (i.e., the maximum pulse wave amplitude is obtained) at the time of previous measurement. The processor 110 determines whether or not the cuff pressure in the depressurization process (i.e., the cuff pressure currently being depressurized) at the time of current blood pressure measurement has reached the extracted cuff pressure.

When the cuff pressure being depressurized has not reached the extracted cuff pressure (NO in step S190), the processor 110 executes step S146. When the cuff pressure being depressurized has reached the extracted cuff pressure (YES in step S190), the processor 110 changes the depressurization speed from the speed Vf to the speed Vg (e.g., 4 mmHg/s) (step S150).

In the example of FIG. 13, the speed change period (i.e., the period Tg) in which the cuff pressure is depressurized at the speed Vg is a period from the time point at which determination is made that the cuff pressure at the time of current measurement has reached the extracted cuff pressure until after elapse of a specified time. Note that in step S190 described above, the processor 110 may extract the cuff pressure at a timing at which the pulse wave amplitude smaller by a predetermined value (alternatively, greater by a predetermined value) than the maximum pulse wave amplitude in the depressurization process at the time of previous measurement is obtained based on the related information. However, the predetermined value is assumed to be a small value.

### Second Modified Example

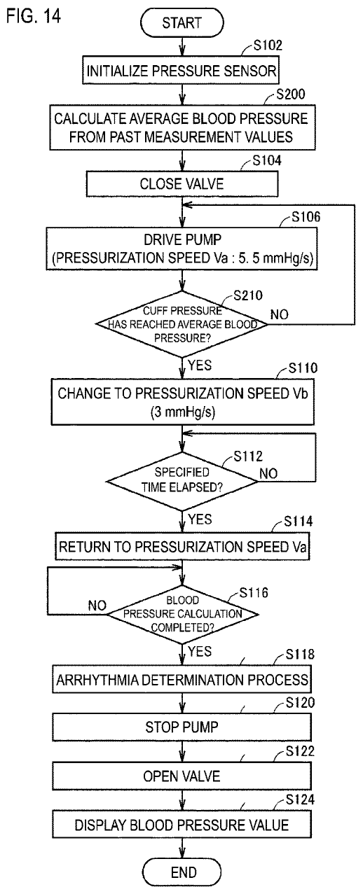

FIG. 14 is a flowchart illustrating a second modified example of a blood pressure measurement process (pressurization measurement method) in the arrhythmia determination mode.

Referring to FIG. 14, the processes in steps S102, S104, S106 and S110 to S124 are similar to the corresponding processes in FIG. 9, and thus the detailed description thereof will not be repeated.

After the process of step S102, the processor 110 calculates the average blood pressure based on the past blood pressure measurement value (step S200). Specifically, the processor 110 reads the systolic blood pressure and the diastolic blood pressure measured at the time of past blood pressure measurement (e.g., at the time of previous measurement) from the memory 51, and calculates the average blood pressure based on the systolic blood pressure and the diastolic blood pressure.

In addition, the processor 110 starts pressurization of the cuff pressure by driving the pump 32 (step S106), and determines whether or not the cuff pressure has reached the average blood pressure calculated in step S200 (step S210). When the cuff pressure has not reached the average blood pressure (NO in step S210), the processor 110 executes step S106. When the cuff pressure has reached the average blood pressure (YES in step S210), the processor 110 changes the pressurization speed from the speed Va to the speed Vb (e.g., 3 mmHg/s) (step S110).

In the example of FIG. 14, the speed change period (i.e., the period Tb) in which the cuff pressure is pressurized at the speed Vb is a period from the time point when determination is made that the cuff pressure has reached the average blood pressure until after elapse of a specified time. Note that in step S210 described above, the processor 110 may determine whether or not the cuff pressure has reached a blood pressure smaller than the average blood pressure by a predetermined pressure (alternatively, greater by a predetermined pressure). However, the predetermined pressure is assumed to be a small value.

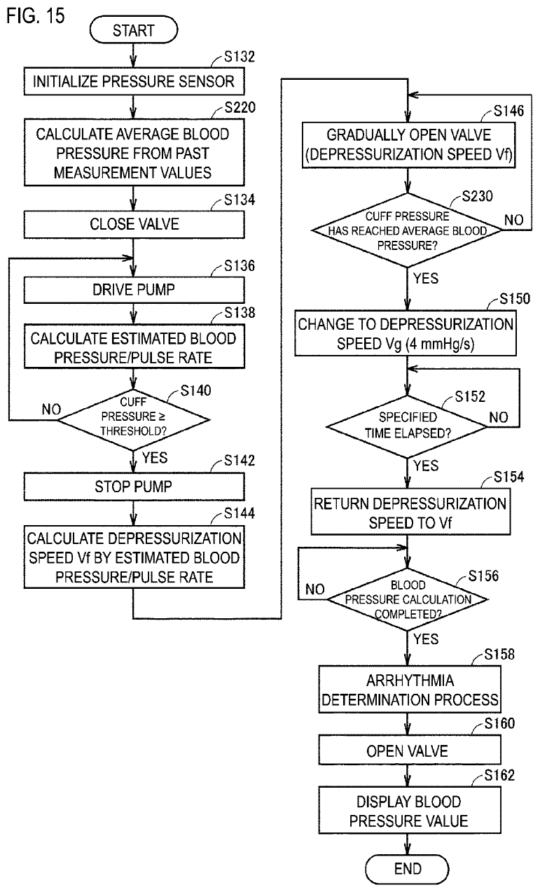

FIG. 15 is a flowchart illustrating a second modified example of a blood pressure measurement process (depressurization measurement method) in the arrhythmia determination mode.

Referring to FIG. 15, the processes in steps S132, S134 to S146, and S150 to S162 are similar to the corresponding processes in FIG. 11, and thus the detailed description thereof will not be repeated.

After the process of step S132, the processor 110 calculates the average blood pressure based on the past blood pressure measurement value (step S220).

Furthermore, after the process of step S146, the processor 110 determines whether or not the cuff pressure being depressurized has reached the average blood pressure calculated in step S220 (step S230). When the cuff pressure has not reached the average blood pressure (NO in step S230), the processor 110 executes step S146. When the cuff pressure has reached the average blood pressure (YES in step S230), the processor 110 changes the depressurization speed from the speed Vf to the speed Vg (e.g., 4 mmHg/s) (step S150).

In the example of FIG. 15, the speed change period (i.e., the period Tg) in which the cuff pressure is depressurized at the speed Vg is a period from the time point when determination is made that the cuff pressure has reached the average blood pressure until after elapse of a specified time. Note that in step S210 described above, the processor 110 may determine whether or not the cuff pressure has reached a blood pressure smaller than the average blood pressure by a predetermined pressure (alternatively, greater by a predetermined pressure). However, the predetermined pressure is assumed to be a small value.

### Third Modified Example

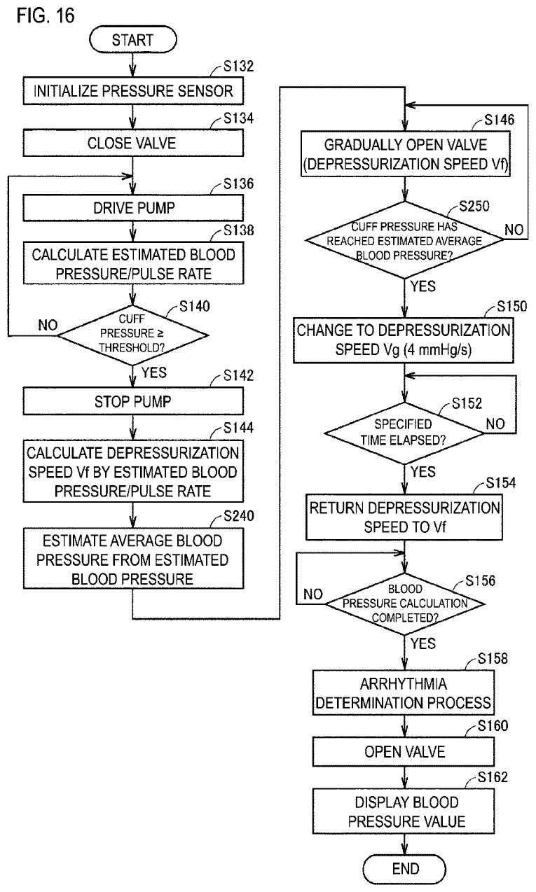

FIG. 16 is a flowchart illustrating a third modified example of a blood pressure measurement process (depressurization measurement method) in the arrhythmia determination mode.

Referring to FIG. 16, the processes in steps S132 to S144, S146, and S150 to S162 are similar to the corresponding processes in FIG. 11, and thus the detailed description thereof will not be repeated.

After the process of step S144, the processor 110 estimates the average blood pressure based on the estimated systolic blood pressure and the estimated diastolic blood pressure calculated in step S138 (step S240). Specifically, the processor 110 calculates the estimated average blood pressure based on the estimated systolic blood pressure and the estimated diastolic blood pressure.

After the process of step S146, the processor 110 determines whether or not the cuff pressure being depressurized has reached the estimated average blood pressure calculated in step S240 (step S250). When the cuff pressure has not reached the estimated average blood pressure (NO in step S250), the processor 110 executes step S146. When the cuff pressure has reached the estimated average blood pressure (YES in step S250), the processor 110 changes the depressurization speed from the speed Vf to the speed Vg (e.g., 4 mmHg/s) (step S150).

In the example of FIG. 16, the speed change period (i.e., the period Tg) in which the cuff pressure is depressurized at the speed Vg is a period from the time point when determination is made that the cuff pressure has reached the estimated average blood pressure until after elapse of a specified time. Note that in step S250 described above, the processor 110 may determine whether or not the cuff pressure has reached a blood pressure smaller than the estimated average blood pressure by a predetermined pressure (alternatively, greater by a predetermined pressure). However, the predetermined pressure is assumed to be a small value.

### Fourth Modified Example

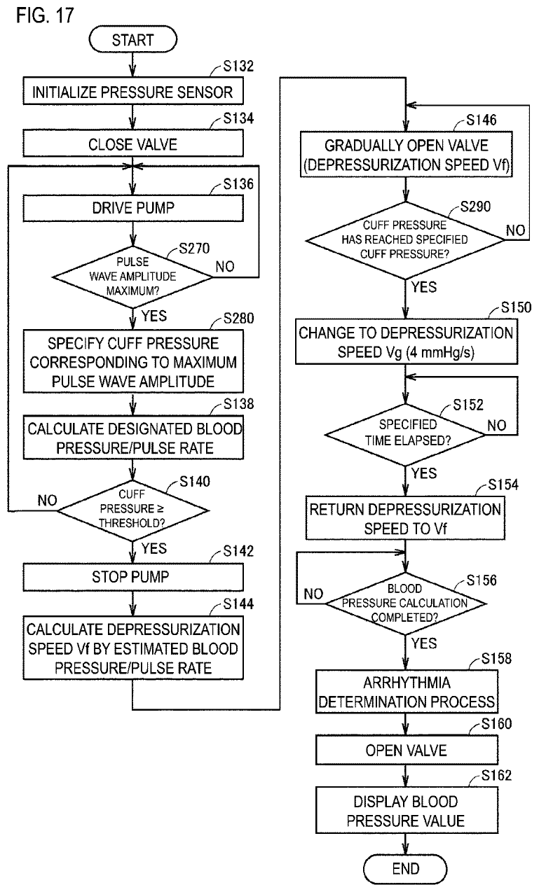

FIG. 17 is a flowchart illustrating a fourth modified example of a blood pressure measurement process (depressurization measurement method) in the arrhythmia determination mode.

Referring to FIG. 17, the processes in steps S132 to S136, S138 to S146, and S150 to S162 are similar to the corresponding processes in FIG. 11, and thus the detailed description thereof will not be repeated.

After the process of step S136, the processor 110 determines whether or not the pulse wave amplitude has is the maximum in the pressurization process (step S270). When the pulse wave amplitude is not the maximum (NO in step S270), the processor 110 continues the pressurization of the cuff pressure by executing the process of step S136. When the pulse wave amplitude is maximum (YES in step S270), the processor 110 specifies the cuff pressure corresponding to the maximum pulse wave amplitude (i.e., the cuff pressure at a timing at which the pulse wave amplitude is the maximum) (step S280), and executes the process of step S138.

After the process of step S146, the processor 110 determines whether or not the cuff pressure being depressurized has reached the cuff pressure specified in step S280 (hereinafter, also referred to as “specific cuff pressure”) (step S290). When the cuff pressure has not reached the specific cuff pressure (NO in step S290), the processor 110 executes step S146. When the cuff pressure has reached the specific cuff pressure (YES in step S290), the processor 110 changes the depressurization speed from the speed Vf to the speed Vg (e.g., 4 mmHg/s) (step S150).

In the example of FIG. 17, the speed change period (i.e., the period Tg) in which the cuff pressure is depressurized at the speed Vg is a period from the time point when determination is made that the cuff pressure has reached the specific cuff pressure until after elapse of a specified time. Note that in step S290 described above, the processor 110 may determine whether or not the cuff pressure has reached a pressure smaller than the specific cuff pressure by a predetermined pressure (alternatively, greater by a predetermined pressure). However, the predetermined pressure is assumed to be a small value.

### Other Embodiments

(1) In the above-described embodiment, the configuration in which the pressurization speed is reduced in the period Tb and the depressurization speed is reduced in the period Tg has been described, but the is not limited to this configuration. For example, a configuration may be adopted to stop the pressurization of the cuff pressure (i.e., set the pressurization speed to zero) in the period Tb, and stop the depressurization of the cuff pressure (i.e., set the depressurization speed to zero) in the period Tg.

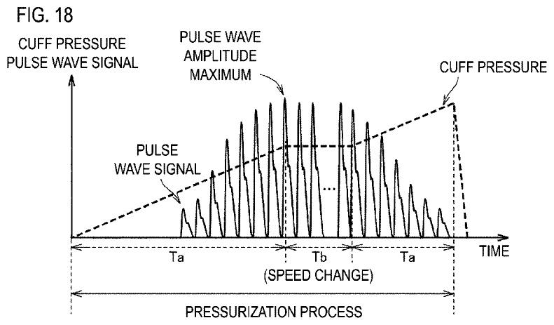

FIG. 18 is a diagram illustrating a correspondence relationship between a pulse wave signal and a cuff pressure at the time of blood pressure measurement (pressurization measurement method) in the arrhythmia determination mode according to another embodiment. Referring to FIG. 18, in the period Tb, the pressurization of the cuff pressure is stopped, and the cuff pressure is maintained. The speed Vb, which is the pressurization speed in the period Tb, is zero.

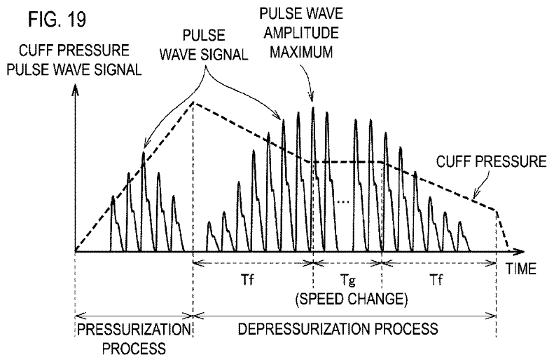

FIG. 19 is a diagram illustrating a correspondence relationship between a pulse wave signal and a cuff pressure at the time of blood pressure measurement (depressurization measurement method) in the arrhythmia determination mode according to another embodiment. Referring to FIG. 19, in the period Tg, the depressurization of the cuff pressure is stopped, and the cuff pressure is maintained. The speed Vg, which is the depressurization speed of the period Tg, is zero.

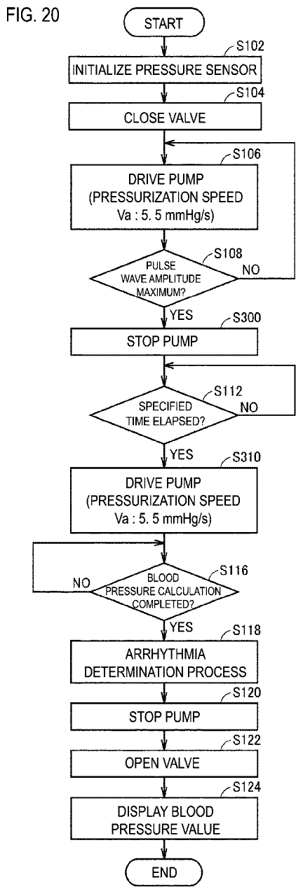

FIG. 20 is a flowchart illustrating a blood pressure measurement process (pressurization measurement method) in the arrhythmia determination mode according to another embodiment. The flowchart of FIG. 20 corresponds to that in which steps S110 and S114 in FIG. 9 are replaced with steps S300 and S310, respectively.

Referring to FIG. 20, the processes in steps S102 to S108, S112, and S116 to S124 are similar to the corresponding processes in FIG. 9, and thus the detailed description thereof will not be repeated.

When the pulse wave amplitude is the maximum (YES in step S108), the processor 110 stops the pump 32 (step S300). As a result, the pressurization of the cuff pressure is stopped, and the cuff pressure is maintained.

The processor 110 determines whether or not a specified time has elapsed since the pump 32 has been stopped (since the cuff pressure starts to be maintained) (step S112). When the specified time has not elapsed (NO in step S112), the processor 110 executes step S112. When the specified time has elapsed (YES in step S112), the processor 110 drives the pump 32 to start pressurization of the cuff 20 at the speed Va (step S310), and executes step S116.

Similarly, steps S110 and S114 in FIG. 12 and FIG. 14 may be replaced with steps S300 and S310, respectively.

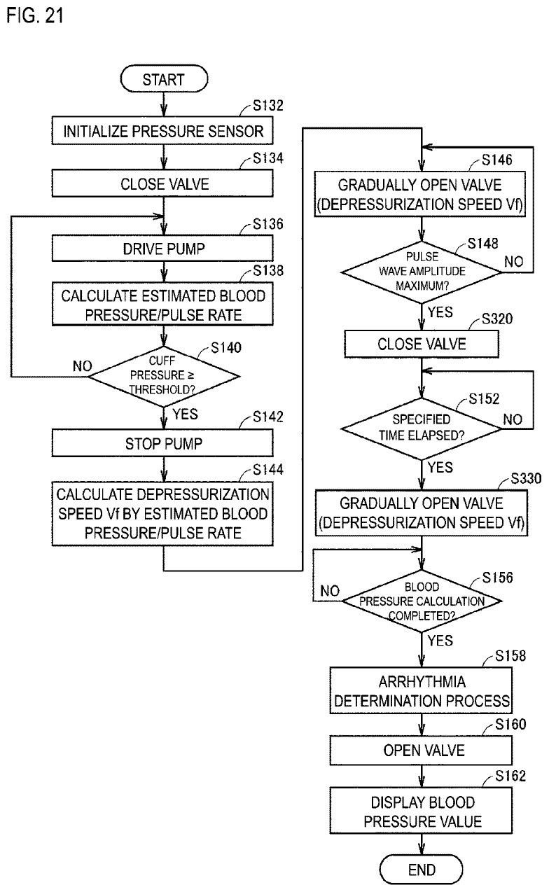

FIG. 21 is a flowchart illustrating a blood pressure measurement process (depressurization measurement method) in the arrhythmia determination mode according to another embodiment. The flowchart of FIG. 21 corresponds to that in which steps S150 and S154 in FIG. 11 are replaced with steps S320 and S330, respectively.

Referring to FIG. 21, the processes in steps S132 to S148, S152, and S156 to S162 are similar to the corresponding processes in FIG. 11, and thus the detailed description thereof will not be repeated.

When the pulse wave amplitude is the maximum (YES in step S148), the processor 110 closes the valve 33 (step S320). As a result, the depressurization of the cuff pressure is stopped, and the cuff pressure is maintained. The processor 110 determines whether or not a specified time has elapsed since the valve 33 has been closed (since the cuff pressure starts to be maintained) (step S152). When the specified time has not elapsed (NO in step S152), the processor 110 executes step S152. When the specified time has elapsed (YES in step S152), the processor 110 performs control to gradually open the valve 33 at the speed Vf (step S330), and executes step S156.

Similarly, steps S150 and S154 in FIG. 13, FIG. 15, FIG. 16, and FIG. 17 may be replaced with steps S320 and S330, respectively.
(2) In the above-described embodiment, a program may be provided that causes a computer to function and execute controls such as those described in the flowcharts described above. Such a program can also be provided as a program product recorded on a non-temporary computer-readable recording medium attached to a computer, such as a flexible disk, a compact disc read only memory (CD-ROM), a secondary storage device, a main storage device, and a memory card. Alternatively, a program may be provided by recording the program on a recording medium such as a hard disk built into a computer. The program may also be provided by download via a network.
(3) The configuration exemplified as the embodiment described above is an example of a configuration of the present invention, and the configuration can be combined with other known technology, and one part thereof may be omitted or modified within the scope not deviating from the gist of the present invention. Furthermore, the processes and configurations described in other embodiments may be employed as appropriate in the embodiments described above.

### Supplementary Notes

As described above, the present embodiments include the following disclosures.

### Configuration 1

A sphygmomanometer (100) comprising: a blood pressure measurement unit (220) configured to measure a blood pressure of a user based on a pulse wave signal in a pressurization process of pressurizing a cuff pressure indicating an inner pressure of a cuff (20) worn on a part of the user to be measured, wherein the blood pressure measurement unit (220) sets a pressurization speed in a predetermined period of the pressurization process to be slower than a pressurization speed in a period other than the predetermined period of the pressurization process, the predetermined period is set based on a timing at which an amplitude of a pulse wave signal during the pressurization process is the maximum or a timing at which the cuff pressure during the pressurization process is an average blood pressure of the user, and a determination unit (230) configured to determine an arrhythmia of the user based on a pulse wave signal in the pressurization process is further provided.

### Configuration 2

The sphygmomanometer (100) according to configuration 1, wherein the blood pressure measurement unit (220) sets, as the predetermined period, a period from a timing at which an amplitude of a pulse wave signal during the pressurization process is the maximum until after elapse of a specified time.

### Configuration 3

The sphygmomanometer (100) according to configuration 1, further comprising a storage unit (250) configured to store related information in which the cuff pressure and the pulse wave signal obtained at the time of past blood pressure measurement of the user are associated with each other, wherein the blood pressure measurement unit (220) extracts the cuff pressure at a timing at which the amplitude of the pulse wave signal in the pressurization process is maximum at the time of past blood pressure measurement of the user based on the related information, and sets, as the predetermined period, a period from a timing at which the cuff pressure in the pressurization process at the time of current blood pressure measurement of the user has reached the extracted cuff pressure until after elapse of a specified time.

### Configuration 4

The sphygmomanometer (100) according to configuration 1, further comprising a storage unit (250) configured to store a systolic blood pressure and a diastolic blood pressure of the user obtained at the time of past blood pressure measurement of the user, wherein the blood pressure measurement unit (220) calculates the average blood pressure based on the systolic blood pressure and the diastolic blood pressure, and sets, as the predetermined period, a period from a timing at which the cuff pressure in the pressurization process at the time of current blood pressure measurement of the user has reached the average blood pressure until after elapse of a specified time.

### Configuration 5

The sphygmomanometer (100) according to any one of configurations 1 to 4, wherein the blood pressure measurement unit (220) sets the pressurization speed in the predetermined period to zero.

### Configuration 6

A sphygmomanometer (100) comprising: a blood pressure measurement unit (220) configured to measure, after a pressurization process of pressurizing a cuff pressure indicating an inner pressure of a cuff (20) worn on a part of the user to be measured up to a pressure greater than a specified pressure, a blood pressure of the user based on a pulse wave signal in a depressurization process of depressurizing the cuff pressure, wherein the blood pressure measurement unit (220) sets a depressurization speed in a predetermined period of the depressurization process to be slower than a depressurization speed in a period other than the predetermined period of the depressurization process, the predetermined period is set based on a timing at which an amplitude of a pulse wave signal during the depressurization process is the maximum or a timing at which the cuff pressure during the depressurization process is an average blood pressure of the user, and a determination unit (230) configured to determine an arrhythmia of the user based on a pulse wave signal in the depressurization process is further provided.

### Configuration 7

The sphygmomanometer (100) according to configuration 6, wherein the blood pressure measurement unit (220) sets, as the predetermined period, a period from a timing at which an amplitude of a pulse wave signal during the depressurization process is the maximum until after elapse of a specified time.

### Configuration 8

The sphygmomanometer (100) according to configuration 6, further comprising a storage unit (250) configured to store related information in which the cuff pressure and the pulse wave signal obtained at the time of past blood pressure measurement of the user are associated with each other, wherein the blood pressure measurement unit (220) extracts the cuff pressure at a timing at which the amplitude of the pulse wave signal in the depressurization process is maximum at the time of past blood pressure measurement of the user based on the related information, and sets, as the predetermined period, a period from a timing at which the cuff pressure in the depressurization process at the time of current blood pressure measurement of the user has reached the extracted cuff pressure until after elapse of a specified time.

### Configuration 9

The sphygmomanometer (100) according to configuration 6, further comprising a storage unit (250) configured to store a systolic blood pressure and a diastolic blood pressure of the user obtained at the time of past blood pressure measurement of the user, wherein the blood pressure measurement unit (220) calculates the average blood pressure based on the systolic blood pressure and the diastolic blood pressure, and sets, as the predetermined period, a period from a timing at which the cuff pressure in the depressurization process at the time of current blood pressure measurement of the user has reached the average blood pressure until after elapse of a specified time.

### Configuration 10

The sphygmomanometer (100) according to configuration 6, wherein in the pressurization process, the blood pressure measurement unit (220) estimates a systolic blood pressure and a diastolic blood pressure of the user, and estimates the average blood pressure based on the estimated systolic blood pressure and diastolic blood pressure, and sets, as the predetermined period, a period from a timing at which the cuff pressure in the depressurization process has reached the average blood pressure until after elapse of a specified time.

### Configuration 11

The sphygmomanometer (100) according to configuration 6, wherein the blood pressure measurement unit (220) specifies a cuff pressure at a timing at which the amplitude of the pulse wave signal during the pressurization process is the maximum, and sets, as the predetermined period, a period from a timing at which the cuff pressure in the depressurization process has reached the specified cuff pressure until after elapse of a specified time.

### Configuration 12

The sphygmomanometer (100) according to any one of configurations 6 to 11, wherein the blood pressure measurement unit (220) sets the depressurization speed in the predetermined period to zero.

### Configuration 13

A method for measuring a blood pressure comprising:
measuring a blood pressure of a user based on a pulse wave signal in a pressurization process of pressurizing a cuff pressure indicating an inner pressure of a cuff (20) worn on a part of the user to be measured, wherein the measuring includes setting a pressurization speed in a predetermined period of the pressurization process to be slower than a pressurization speed in a period other than the predetermined period of the pressurization process, the predetermined period is set based on a timing at which an amplitude of a pulse wave signal during the pressurization process is the maximum or a timing at which the cuff pressure during the pressurization process is an average blood pressure of the user, and the method further includes determining an arrhythmia of the user based on a pulse wave signal in the pressurization process.

### Configuration 14

A method for measuring a blood pressure comprising:
measuring, after a pressurization process of pressurizing a cuff pressure indicating an inner pressure of a cuff (20) worn on a part of the user to be measured up to a pressure greater than a specified pressure, a blood pressure of the user based on a pulse wave signal in a depressurization process of depressurizing the cuff pressure, wherein the measuring includes setting a depressurization speed in a predetermined period of the depressurization process to be slower than a depressurization speed in a period other than the predetermined period of the depressurization process, the predetermined period is set based on a timing at which an amplitude of a pulse wave signal during the depressurization process is the maximum or a timing at which the cuff pressure during the depressurization process is an average blood pressure of the user, and the method further includes determining an arrhythmia of the user based on a pulse wave signal in the depressurization process.

The embodiments disclosed herein are illustrative in all respects and are not intended as limitations. The scope of the present invention is indicated not by the descriptions above but by the claims and includes all meaning equivalent to the scope and changes within the scope.

### REFERENCE SIGNS LIST

10 Main body, 20 Cuff, 22 Fluid bag, 30 Air-system component, 31 Pressure sensor, 32 Pump, 33 Valve, 50 Display, 51 Memory, 52 Operation unit, 52A Measurement switch, 52B Mode selection switch, 53 Communication interface, 54 Power source unit, 100 Sphygmomanometer, 110 Processor, 210 Mode setting unit, 220 Blood pressure measurement unit, 230 Determination unit, 240 Output control unit, 250 Storage unit, 310 A/D conversion circuit, 320 Pump drive circuit, 330 Valve drive circuit.

1. A sphygmomanometer comprising:
a blood pressure measurement unit configured to measure a blood pressure of a user based on a pulse wave signal in a pressurization process of pressurizing a cuff pressure indicating an inner pressure of a cuff worn on a part of the user to be measured, wherein
the blood pressure measurement unit sets a pressurization speed in a predetermined period of the pressurization process to be slower than a pressurization speed in a period other than the predetermined period of the pressurization process,
the predetermined period is set based on a timing at which an amplitude of a pulse wave signal during the pressurization process is maximum or a timing at which the cuff pressure during the pressurization process is an average blood pressure of the user, and
the sphygmomanometer further includes a determination unit configured to determine an arrhythmia of the user based on a pulse wave signal in the pressurization process.

2. The sphygmomanometer according to claim 1, wherein
the blood pressure measurement unit sets, as the predetermined period, a period from a timing at which the amplitude of the pulse wave signal during the pressurization process is maximum until after elapse of a specified time.

3. The sphygmomanometer according to claim 1, further comprising:
a storage unit configured to store related information in which the cuff pressure and the pulse wave signal obtained at the time of past blood pressure measurement of the user are associated with each other, wherein
the blood pressure measurement unit,
extracts the cuff pressure at a timing at which the amplitude of the pulse wave signal in the pressurization process is maximum at the time of past blood pressure measurement of the user based on the related information, and
sets, as the predetermined period, a period from a timing at which the cuff pressure in the pressurization process at the time of current blood pressure measurement of the user reaches the extracted cuff pressure until after elapse of a specified time.

4. The sphygmomanometer according to claim 1, further comprising:
a storage unit configured to store related information in which the cuff pressure and the pulse wave signal obtained at the time of past blood pressure measurement of the user are associated with each other; wherein
the blood pressure measurement unit,
extracts the cuff pressure at a timing at which the amplitude of the pulse wave signal in the pressurization process becomes is a maximum at the time of past blood pressure measurement of the user based on the related information, and
sets, as the predetermined period, a period from a timing at which the cuff pressure in the pressurization process at the time of current blood pressure measurement of the user reaches the extracted cuff pressure until after elapse of a specified time.

5. The sphygmomanometer according to claim 1, wherein
the blood pressure measurement unit sets the pressurization speed in the predetermined period to zero.

6. A sphygmomanometer comprising:
a blood pressure measurement unit configured to measure, after a pressurization process of pressurizing a cuff pressure indicating an inner pressure of a cuff worn on a part of the user to be measured up to a pressure greater than a specified pressure, a blood pressure of the user based on a pulse wave signal in a depressurization process of depressurizing the cuff pressure, wherein
the blood pressure measurement unit sets a depressurization speed in a predetermined period of the depressurization process to be slower than a depressurization speed in a period other than the predetermined period of the depressurization process,
the predetermined period is set based on a timing at which an amplitude of a pulse wave signal during the depressurization process is maximum or a timing at which the cuff pressure during the depressurization process is an average blood pressure of the user, and
the sphygmomanometer further includes a determination unit configured to determine an arrhythmia of the user based on a pulse wave signal in the depressurization process.

7. The sphygmomanometer according to claim 6, wherein
the blood pressure measurement unit sets, as the predetermined period, a period from a timing at which the amplitude of the pulse wave signal during the depressurization process is maximum until after elapse of a specified time.

8. The sphygmomanometer according to claim 6, further comprising:
a storage unit configured to store related information in which the cuff pressure and the pulse wave signal obtained at the time of past blood pressure measurement of the user are associated with each other, wherein
the blood pressure measurement unit,
extracts the cuff pressure at a timing at which the amplitude of the pulse wave signal in the depressurization process is maximum at the time of past blood pressure measurement of the user based on the related information, and
sets, as the predetermined period, a period from a timing at which the cuff pressure in the depressurization process at the time of current blood pressure measurement of the user reaches the extracted cuff pressure until after elapse of a specified time.

9. The sphygmomanometer according to claim 6, further comprising:
a storage unit configured to store a systolic blood pressure and a diastolic blood pressure of the user obtained at the time of past blood pressure measurement of the user, wherein
the blood pressure measurement unit,
calculates the average blood pressure based on the systolic blood pressure and the diastolic blood pressure, and
sets, as the predetermined period, a period from a timing at which the cuff pressure in the depressurization process at the time of current blood pressure measurement of the user reaches the average blood pressure until after elapse of a specified time.

10. The sphygmomanometer according to claim 6, wherein
the blood pressure measurement unit,
in the pressurization process, estimates a systolic blood pressure and a diastolic blood pressure of the user, and estimates the average blood pressure based on the estimated systolic blood pressure and diastolic blood pressure, and
sets, as the predetermined period, a period from a timing at which the cuff pressure in the depressurization process reaches the average blood pressure until after elapse of a specified time.

11. The sphygmomanometer according to claim 6, wherein
the blood pressure measurement unit,
specifies a cuff pressure at a timing at which the amplitude of the pulse wave signal during the pressurization process is the maximum, and
sets, as the predetermined period, a period from a timing at which the cuff pressure in the depressurization process reaches the specified cuff pressure until after elapse of a specified time.

12. The sphygmomanometer according to claim 6, wherein
the blood pressure measurement unit sets the depressurization speed in the predetermined period to zero.

13. method for measuring a blood pressure comprising:
measuring a blood pressure of a user based on a pulse wave signal in a pressurization process of pressurizing a cuff pressure indicating an inner pressure of a cuff worn on a part of the user to be measured, wherein
the measuring includes setting a pressurization speed in a predetermined period of the pressurization process to be slower than a pressurization speed in a period other than the predetermined period of the pressurization process,
the predetermined period is set based on a timing at which an amplitude of a pulse wave signal during the pressurization process is maximum or a timing at which the cuff pressure during the pressurization process is an average blood pressure of the user, and
the method further includes determining an arrhythmia of the user based on a pulse wave signal in the pressurization process.

14. A method for measuring a blood pressure comprising:
measuring, after a pressurization process of pressurizing a cuff pressure indicating an inner pressure of a cuff worn on a part of the user to be measured up to a pressure greater than a specified pressure, a blood pressure of the user based on a pulse wave signal in a depressurization process of depressurizing the cuff pressure, wherein
the measuring includes setting a depressurization speed in a predetermined period of the depressurization process to be slower than a depressurization speed in a period other than the predetermined period of the depressurization process,
the predetermined period is set based on a timing at which an amplitude of a pulse wave signal during the depressurization process is maximum or a timing at which the cuff pressure during the depressurization process is an average blood pressure of the user, and
the method further includes determining an arrhythmia of the user based on a pulse wave signal in the depressurization process.
